(19) United States
(12) Patent Application Publication
(10) Pub. No.: US 2024/0219253 A1
(43) Pub. Date: Jul. 4, 2024

(54) Adaptation Device for Coupling at least One Sensor to a Tube Shell Wall of a Tube for Fluid Measurement, and Sensor Device Having an Adaptation Device of this Type

(71) Applicants: Raumedic AG, Münchberg (DE); REHAU Industries SE & Co. KG, Rehau (DE)

(72) Inventors: Ricardo Ehrenpfordt, Zwickau (DE); Martin Stöcker, Helmbrechts (DE); Tobias Festel, Sparneck (DE); Thomas Ruhland, Bayreuth (DE)

(21) Appl. No.: 18/560,748

(22) PCT Filed: May 11, 2022
(86) PCT No.: PCT/EP2022/062776

§ 371 Date: Nov. 14, 2023

(30) Foreign Application Priority Data
May 17, 2021 (DE) 10 2021 204 971.0

(51) Int. Cl. G01L 19/00; A61M 1/36; G01K 1/14

(52) U.S. Cl. CPC G01L 19/0038; G01K 1/14; A61M 1/36224; A61M 2205/0216; G01L 2019/0053

(57) ABSTRACT

An adaptation device is used for coupling at least one sensor to a tube shell wall of a tube for measuring a property of a fluid conveyed through the tube. The adaptation device has an adapter passage opening for providing a fluid connection via a tube shell wall passage opening between a tube lumen and the sensor. A sensor receptacle is used to accommodate the sensor in the region of the adapter passage opening. An adapter sealing portion extends around the adapter passage opening for circumferential sealing of the adaptation device around the adapter passage opening with respect to the tube shell wall. This results in an adaptation device which makes sensors which are basically suitable for measuring a fluid property of fluid conveyed through a tube accessible for such fluid measurement within a sensor device.

### CROSS-REFERENCE TO RELATED APPLICATIONS

The present patent application claims the priority of German patent application DE 10 2021 204 971.0, the contents of which are incorporated herein by making reference thereto.

### BACKGROUND OF THE INVENTION

### Field of the Invention

The invention relates to an adaptation device for coupling at least one sensor to a tube shell wall of a tube for measuring a property of a fluid conveyed through the tube. Furthermore, the invention relates to a sensor device comprising such an adaptation device.

### Description of Related Art

A sensor device for measuring a property of a fluid conveyed through a tube is known from EP 3 142 724 B1. Furthermore, pressure sensors are known, for example, which in principle can be used in the context of such a fluid measurement, for example the sensor type “NovaSensor NPC-100” (cf. data sheet 04/2018). DE 10 2008 030 802 A1 discloses a sensor receptacle for fastening a sensor to a tube. DE 20 2020 106 224 U1 discloses a sensor system.

### SUMMARY

It is an object of the present invention to make sensors, which are basically suitable for fluid property measurement of fluid conveyed through a tube, accessible to such fluid measurement within a sensor device.

This object is achieved according to the invention by an adaptation device for coupling at least one sensor to a tube shell wall of a tube for measuring a property of a fluid conveyed through the tube that includes a base body, an adapter passage opening for providing a fluid connection via a tube shell wall passage opening between a tube lumen and the sensor, a sensor receptacle for accommodating the sensor in the region of the adapter passage opening, and an adapter sealing portion extending around the adapter passage opening for sealing the adaptation device with respect to the tube shell wall in a manner surrounding the adapter passage opening with the adaptation device including a mounting structure for supporting a sensor housing, the mounting structure including a plurality of mounting strips clamped between parts and/or between components of the sensor housing.

The adaptation device can be made entirely of plastics, for example of silicone. The adaptation device may be formed integrally. The adaptation device may be designed such that accommodation of the at least one sensor takes place by adhesive bonding and/or welding. The adaptation device may be designed such that the adapter sealing portion is formed by adhesive material and/or by welding. Shrinkage of such adhesive material may be used to produce and/or improve a sealing effect. The mounting structure has proven to securely hold the adaptation device to a sensor housing. The mounting structure comprises a plurality of mounting strips. These mounting strips are clamped between parts and/or between components of the sensor housing.

An adaptable design of the base body of the adaptation device where the base body of the adaptation device is designed to be adaptable to different outer diameters of the tube shell wall increases the possibilities of application of the adaptation device. The adaptation device can then be used with tubes of different tube diameters.

A base body design with a soft component and a hard component where the base body in a region radially adjacent to the tube shell wall is made of a plastic soft component having a smaller Shore hardness compared to a plastic hard component of the base body, which is present in a radially distant region of the base body has proven to increase flexibility of use. The plastic soft component can be used for an adaptable design of the base body of the adaptation device and/or for sealing with respect to the tube shell wall and/or with respect to at least a housing part.

Adaptation recesses in a region radially adjacent to the tube shell wall which permit circumferential adaptation of the base body to the tube shell wall also permit adaptability of the base body of the adaptation device to different tube diameters. In this case, a hard/soft component design of the base body is not required. The adaptation recesses may be designed as wedge-shaped recesses. The recesses can taper radially outwardly.

Embodiments of the adaptation recesses in a region radially adjacent to the tube shell wall which are distributed in the circumferential direction and/or distributed in the axial longitudinal direction in the base body permit adaptation to the tube shell wall. In particular, a tube circumferential adaptation and/or an adaptation to various curvatures of a tube guide can be rendered possible thereby. This permits, for example, decoupling in particular of a result of a pressure measurement via the sensor adapted to be accommodated by way of the adaptation device from a deflection of the tube at the location of the adaptation device.

A positioning protrusion for presetting a position of the adaptation device relative to the tube shell wall by cooperating with at least one positioning recess in the tube shell wall, which is designed to be complementary to the positioning protrusion, permits secure, exact positioning in particular of the adapter passage opening relative to the tube shell wall passage opening. The adaptation device may include a plurality of such positioning protrusions. The at least one positioning protrusion can be designed as a positioning pin. The at least one positioning protrusion can be designed as a positioning sleeve, which in particular also presets the adapter passage opening. Insofar as several positioning protrusions of the adaptation device are present, the tube shell wall has corresponding positioning recesses complementary thereto.

A force measuring structure for measuring a mechanical stress, in particular a mechanical deformation acting on a base body of the adaptation device permits moni-toring of mechanical stress, in particular deformation, acting on the base body of the adaptation device. This can be used for calibration and/or correction purposes or also for warning purposes if, in particular, a permissible mechanical stress is exceeded due to excessive tube deflection, for example. Measurement errors and/or measurement drifts of the at least one sensor can thus be detected and compensated for, if neces-sary.

At least one additional sensor receptacle permits the use of several sensors with the adaptation device. The additional sensor receptacle need not have access to the adapter passage opening. The sensor receptacles may be configured such that a plurality of sensors carried by a common circuit board are accommodated by the adaptation device. The sensor receptacle may be of such a size that an electronic unit for control-ling the at least one sensor can additionally be accommodated by the adaptation device, in particular a processor, for example a microcontroller.

A connector receptacle for an electrical connector for electrical connection of the at least one sensor to an external component facilitates connection of the at least one sensor to an external component, for example to a control/regulation unit for measurement control.

A sensor passage opening for coupling a sensor to the tube shell wall of the tube allows, for example, good thermal contact of a temperature sensor to the fluid conveyed in the tube and/or to the tube wall.

The advantages of a sensor device that includes an adaptation device, at least one sensor accommodated in the at least one sensor receptacle of the adaptation device, and a sensor housing accommodating the at least one sensor correspond to those al-ready explained above with reference to the adaptation device. The at least one sensor may be a pressure sensor, a temperature sensor, a flow sensor, or an optical sensor. Several such sensors can also be accommodated in any combination in corresponding receptacles of the adaptation device. The sensor device is particularly suitable for ECMO application.

Insofar as a pressure sensor is used as the sensor of the sensor device, this sensor can be configured such that it can measure both a negative pressure and a positive pressure. The sensor can cover a pressure measurement range between −200 mmHg to 700 mmHg. Depending on the design of the pressure sensor, this pressure measurement range may also cover the range between −50 mmHg and 300 mmHg.

The adaptation device may include structures for receiving further components such as optical windows or membrane structures.

A design of the sensor device in that the sensor housing is designed as a multi-part housing and the mounting structure is designed such that it simultaneously effects sealing with respect to at least one of the housing parts via an adapter housing seal facilitates sealing, on the one hand, of an interior space of the sensor housing to the outside and/or of the adaptation device around the adapter passage opening with respect to the tube shell wall.

### BRIEF DESCRIPTION OF THE DRAWINGS

Embodiments of the invention will be explained in more detail below with reference to the drawings, wherein:

FIG. 1 shows a perspective view of a sensor device comprising a sensor unit cou-pled to a tube shell wall of a tube via an adaptation device;

FIG. 2 shows a view from viewing direction II in FIG. 1;

FIG. 3 shows a view from viewing direction III in FIG. 1;

FIG. 4 shows a longitudinal cross-sectional view of this sensor device and a tube section of the tube according to line IV-IV in FIG. 3;

FIG. 5 shows a transverse cross-sectional view of the sensor device and the tube according to line V-V in FIG. 4;

FIG. 6 shows a perspective view corresponding to FIG. 1 with the housing cover removed so that details of the adaptation device and the sensor unit are visible;

FIG. 7 shows a perspective view of the assembly according to FIG. 6 from viewing direction VII, in which a connector for electrical connection of the sensor unit to an external component is also omitted;

FIG. 8 shows the assembly in a view similar to FIG. 7, with the sensors of the sensor unit being omitted as well;

FIG. 9 shows a view similar to FIG. 8, illustrating only a lower housing half-shell of the sensor device;

FIG. 10 shows a perspective view of the housing cover of the sensor device;

FIG. 11 shows a perspective view of the adaptation device, as seen obliquely from below, i.e. from the viewpoint of the tube;

FIG. 12 shows the adaptation device, as seen obliquely from above; and

FIG. 13 shows the lower housing half-shell of the sensor device.

### DETAILED DESCRIPTION OF THE PREFERRED EMBODIMENTS

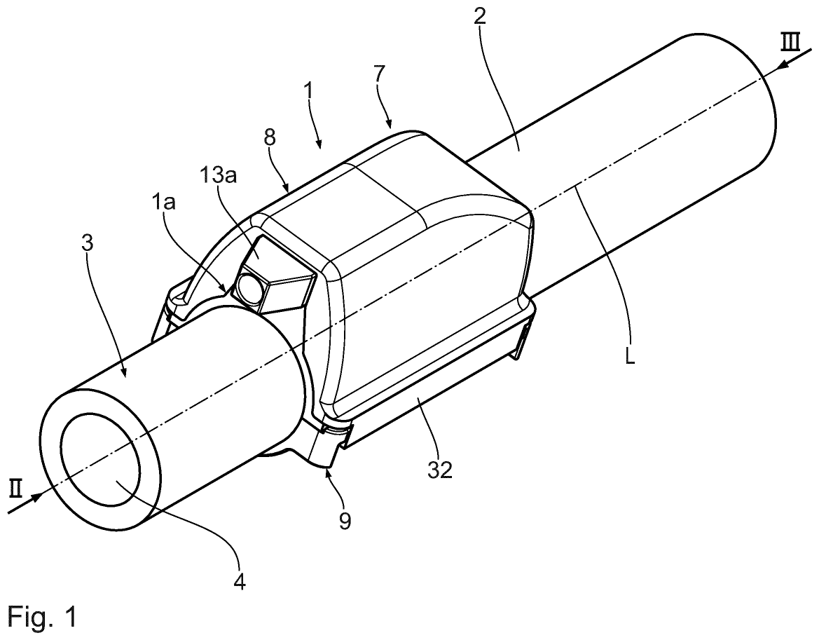

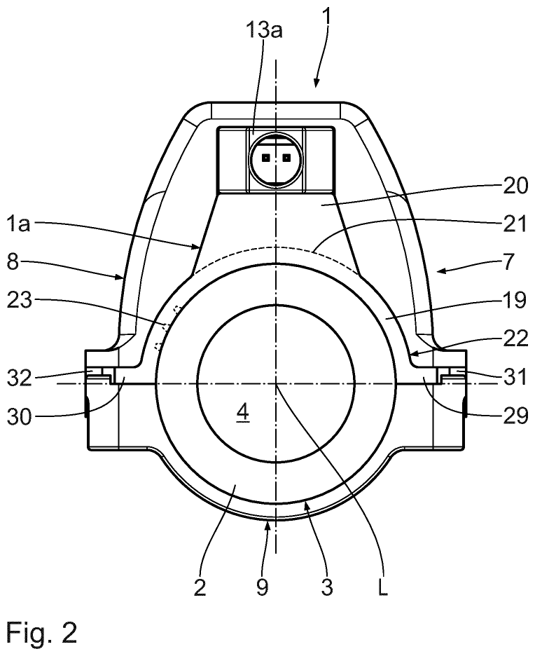

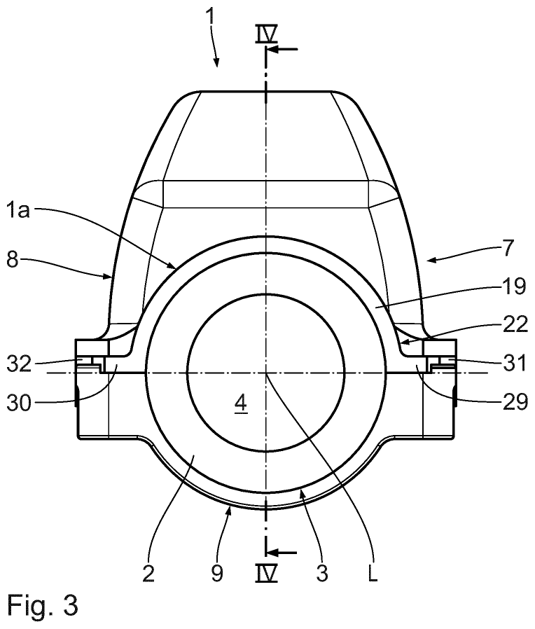

FIGS. 1 to 5 show overall views or cross-sectional views of a sensor device 1 comprising an adaptation device 1a (cf. e.g. FIG. 2) for coupling at least one sensor to a tube shell wall 2 of a tube 3, which is shown in sections in each of the drawings. The tube 3 is a plastic tube, in particular for a medical application. The tube 3 may be an ECC tube (Extra Corporal Circulation, circulation outside the body).

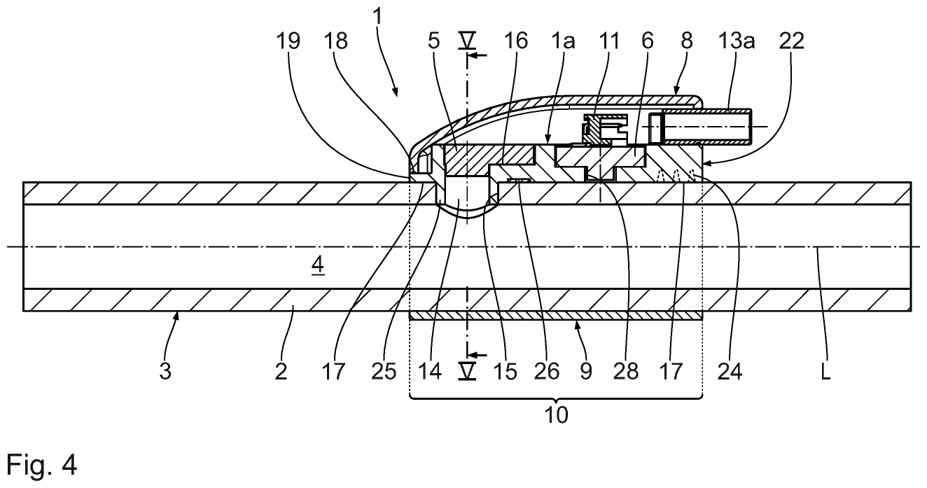

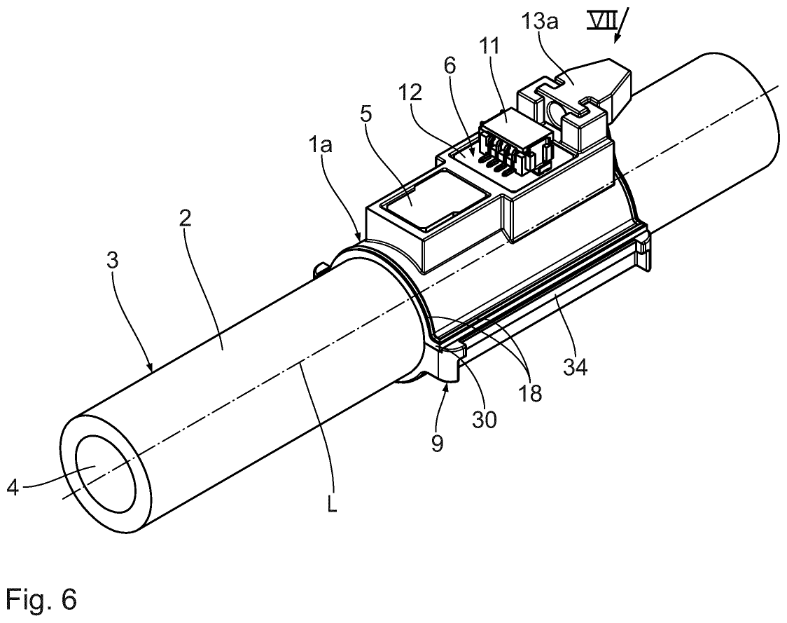

The at least one sensor is part of a sensor unit for measuring a property of a fluid conveyed through a lumen 4 of the tube 3. The adaptation device 1a carries a pressure sensor 5 and a temperature sensor 6 (cf. e.g. FIG. 4). These sensors 5, 6 are part of the sensor unit of the adaptation device 1a. With respect to the sensor 6, a circuit board of this sensor is facing the viewer, for example in FIG. 6, which carries the temperature sensor proper on its side oriented toward the tube shell wall 2. The temperature sensor may be an SMD component or a wired component.

The sensor device 1 has a multi-part sensor housing 7 with a housing cover 8 and a lower housing part 9 designed as a half shell. The sensor housing encloses, on the one hand, the sensor unit with the adaptation device 1a and, on the other hand, an axial tube section 10 of the tube 3. The sensor unit may also include a controller or processor which may have a data memory (not shown in the figures). The connector 1, on the one hand, and the temperature sensor 6, on the other hand, are soldered to both sides of a circuit board 12 of the sensor unit, the temperature sensor 6 facing the tube shell wall 2 in the sensor housing 7. The connector 11 is used for contacting the supply line. In particular, the circuit board 12 is in contact with the pressure sensor 5 via wires, which is not shown in the drawings. Alternatively or additionally, the pressure sensor 5 may be in contact with the connector 11. Alternatively, the pressure sensor 5, the temperature sensor and the connector 11 may be positioned on a common circuit board.

Each of the circuit boards associated with one of the sensors 5, 6, or the common circuit board, may have at least one further electronic component, e.g. a controller, in particular a microcontroller. The controller may then be in electrical contact with the sensors 5 or 6 as well as with the connector 11.

In an embodiment of the sensor unit not illustrated, the two sensors 5, 6 may also be arranged on a common circuit board.

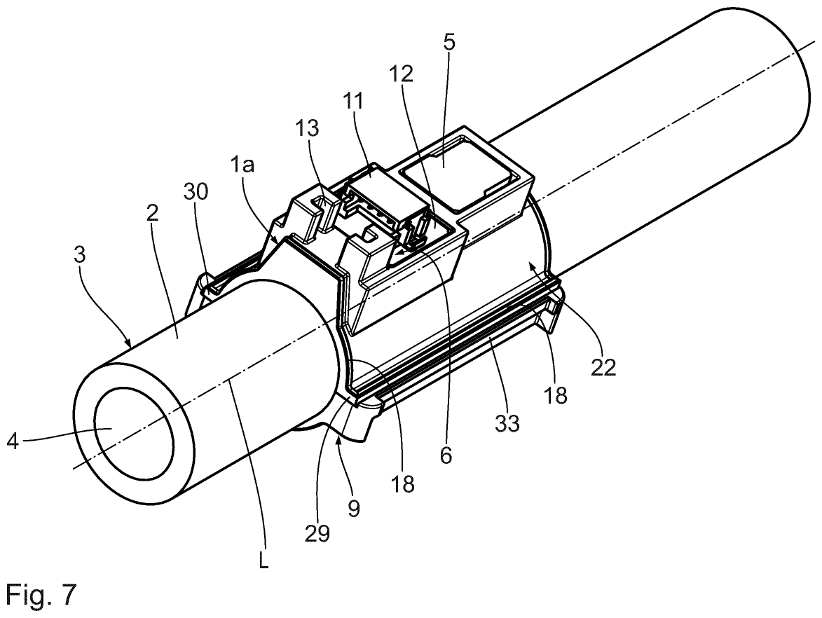

The adaptation device 1a has a connector receptacle 13 (cf. e.g. FIG. 7) for accommodating a mechanical connector 13a, e.g. a cable kink protection (cf. e.g. FIG. 6). Alternatively, the connector receptacle 13 can accommodate an electrical connector (cf. 11) for electrically connecting the sensor unit to the sensors 6, 7 and an external component which may be, for example, a control unit for the sensor device 1 and an electrical supply component. The electrical connector 11 may thus provide, on the one hand, a power supply and, on the other hand, a data connection between the sensor unit and the external component, and may be positioned on the circuit board or in the connector receptacle 13. Such an electrical connector of the type of electrical connector 11 may be in the form of a connector plug. The electrical connector can be attached directly to a housing wall, in particular of the housing cover 8, for example on the face end side where the cable kink protection 13a is shown in FIG. 1. The electrical connector can be designed as a D-sub plug, for example as a 9-pin plug. Alternatively, the electrical connector may be designed as a corresponding female connector or socket.

A controller, for example a microcontroller, of the adaptation device 1a may have a digital serial interface.

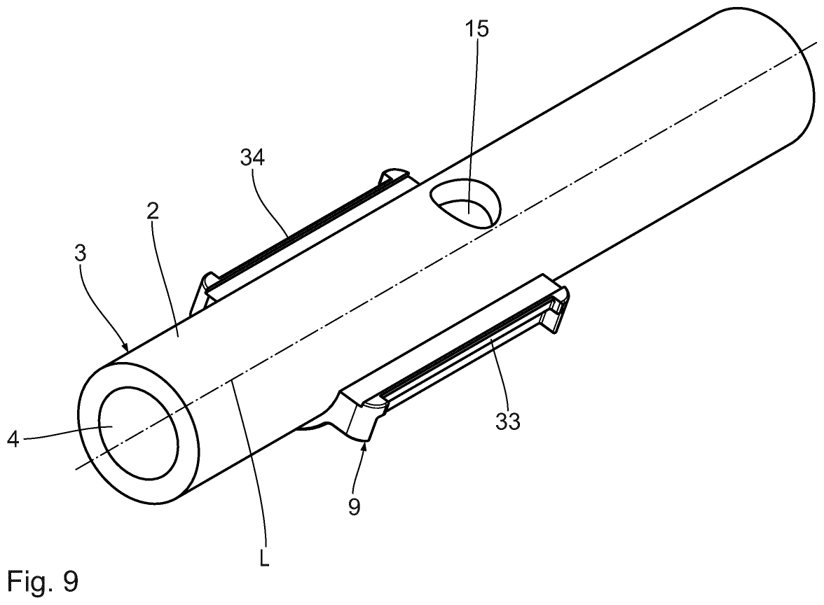

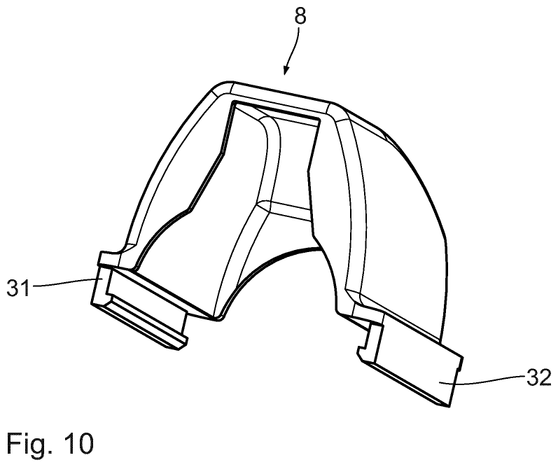

The adaptation device 1a has an adapter passage opening 14 (cf. e.g. FIGS. 4, 5 and 8) for providing a fluid connection via a tube shell wall passage opening 15 (cf. e.g. FIG. 9) between the tube lumen 4 and the pressure sensor 5 of the sensor unit of the adaptation device 1a.

The adaptation device 1a further has a sensor receptacle 16 for accommodating the pressure sensor 5 in the region of the adapter passage opening 14.

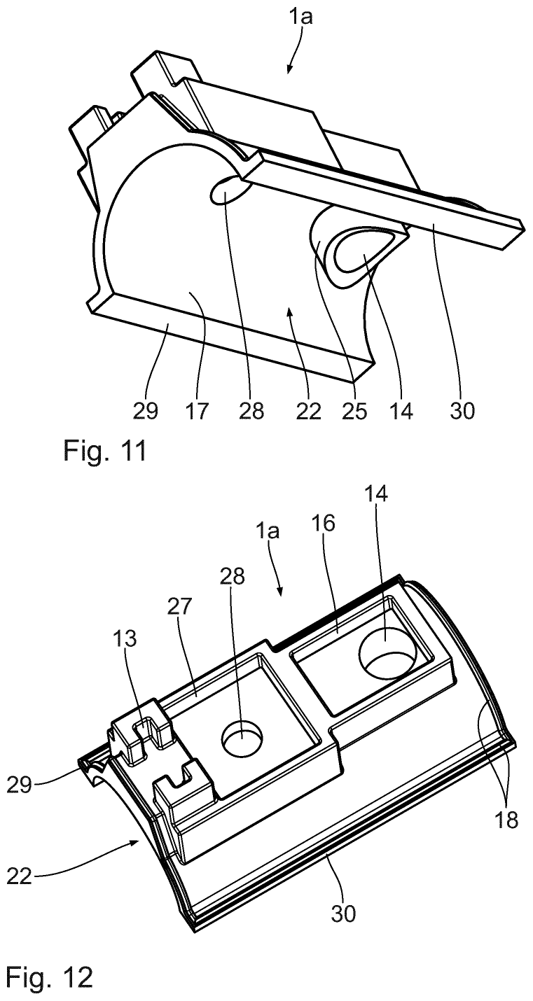

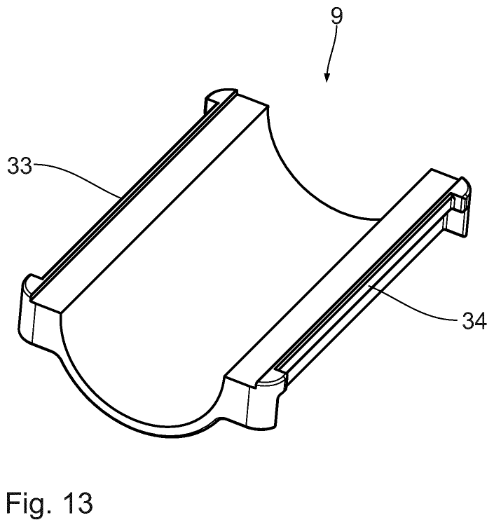

Furthermore, the adaptation device 1a has an adapter sealing portion 17 extending around the adapter passage opening 14 for sealing the adaptation device 1a with respect to the tube shell wall 2 in the circumferential area around the adapter passage opening 14. The latter is formed by face-to-face abutting contact of the adaptation device 1a against the tube shell wall 2 in the area around the tube shell wall passage opening 15 as well as around the adapter passage opening 14 (cf. FIG. 11).

The adaptation device 1a also comprises an additional adapter housing seal 18, which surrounds the adapter passage opening 14 and faces the housing cover 8, for sealing the adaptation device 1a with respect to the housing cover 8 in a manner surrounding the adapter passage opening 14. Fluid can thus not escape from the lumen 4 via the tube shell wall passage opening 15 into a surrounding area around the sensor housing 7. The adapter housing seal 18 may be a gasket, in particular an O-ring seal, a sealing bead, or an adhesive seam.

The adapter sealing portion 17 may be formed by a plastic soft component of the adaptation device 1a, which has a smaller Shore hardness in a region of the adaptation device 1a radially adjacent to the tube shell wall 2 as compared to another plastic hard component of the adaptation device 1a. In FIG. 2, for a first, corresponding 2K (two-component) design of the adaptation device 1a, a transition region between such a plastic soft component 19 and a radially outer plastic hard component 20 is indicated by a dashed line at 21.

The plastic soft component 19 can have a Shore A hardness in the range between 30 and 60, for example in the range between 50 and 60 or also in the range between 30 and 50.

Material for the plastic soft component 19 may be silicone. As an alternative to silicone, it is also possible to use a different material for the plastic soft component 19 with a corresponding Shore hardness.

The plastic hard component 20 may have a Shore A hardness in the range between 60 and 98, in particular in the range between 80 and 98 or also between 60 and 80.

The sensor housing 7 and/or the plastic hard component 20 may be made of silicone, of PUR (polyurethane) or of ABS (acrylonitrile butadiene styrene copolymer).

The plastic design of the adaptation device 1a in the area abutting the tube 3 can be such that a base body 22 of the adaptation device 1a is designed to be adaptable to different outer diameters of the tube shell wall of the respective tube used.

The plastic soft component 19 may be formed as an entire half-shell portion of the base body 22, which abuts the tube shell wall 2 and is integrally molded on a supporting portion of the base body 22 for the sensor unit, which is formed as the plastic hard component.

Alternatively, for adaptability of the base body 22 of the adaptation device 1a to different outer diameters of the tube shell wall 2, the base body 22 may have adaptation recesses 23 in an area radially adjacent to the tube shell wall 2, which are indicated at three locations in FIG. 2. The adaptation recesses 23 extend in the longitudinal direction of the base body 22, i.e. parallel to the tube axis, and have a wedge-shaped cross-section which tapers outwardly in the radial direction of the tube 3 to a recess base which forms a roof edge of the wedge. Via these adaptation recesses 23, circumferential adaptation of the base body 22 to the tube shell wall 2 can be rendered possible. The adaptation recesses 23 are formed in the base body so as to be distributed in the circumferential direction around the tube 3.

Corresponding adaptation recesses 24 can also be provided in the base body 22 of the adaptation device 1a distributed in the axial longitudinal direction, as indicated at three locations in FIG. 4. These further adaptation recesses 24 extend in the circumferential direction around the longitudinal axis L of the tube and also have a wedge-shaped cross-section which tapers radially outwardly.

The base body 22 of the adaptation device 1a has at least one positioning protrusion 25 for presetting a position of the adaptation device 1a relative to the tube shell wall 2 by cooperating with at least one positioning recess in the tube shell wall 2, which is designed to be complementary to the positioning protrusion 25. This positioning is such that the adapter passage opening 14 is aligned with the tube shell wall passage opening 15. In the embodiment illustrated in the drawings, the positioning recess of the tube shell wall 2 is formed by the tube shell wall passage opening 15. The positioning protrusion 25 is formed as a positioning sleeve of the adaptation device 1a, which is integrally molded on the base body 22 and projects beyond the base body 22 and which at the same time defines the adapter passage opening 14.

Depending on the design of the adaptation device 1a, a plurality of positioning protrusions may also be formed, for example positioning pins which engage in positioning recesses, which are complementary thereto and are formed in the tube shell wall 2, as soon as correct positioning of the adaptation device 1a with respect to the tube shell wall 2 in the axial direction and in the circumferential direction is achieved, so that the adapter passage opening 14 is aligned with the tube shell wall passage opening 15.

Such positioning protrusions/positioning recesses may also have a sealing func-tion of the adaptation device 1a with respect to the tube shell wall 2.

The adaptation device 1a may further comprise at least one force measuring structure 26 (cf. FIG. 4). The force measuring structure 26 is positioned in the region of an abutment of the base body 22 against the tube shell wall 2 and is used to measure stress acting on the base body 22 of the adaptation device 1a during its use in fluid property measurement. Measurement data of the force measurement structure 26 can be used for calibration and/or correction of measurement data of the pressure sensor 5.

The force measuring structure 26 can be designed as a strain gauge.

The force measuring structure 26 is in signal connection with the processor 11 or the electrical connector 13a or one of the other sensors 5, 6 in a manner not shown.

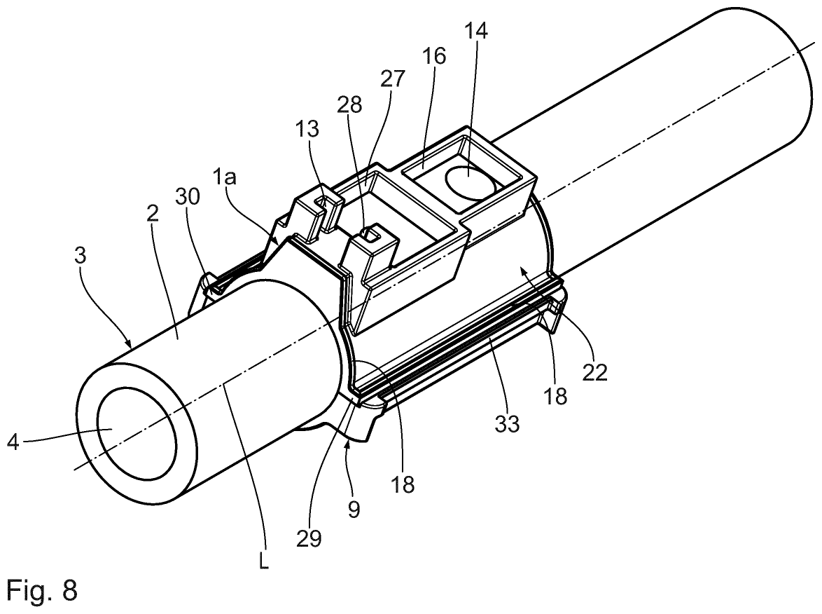

In addition to the sensor receptacle 16 for accommodating the pressure sensor 5, the adaptation device 1a comprises another sensor receptacle 27 (cf. FIG. 8) for accommodating the temperature sensor 6. On the bottom side, the sensor receptacle 27 has a sensor passage opening 28 (cf. e.g. FIGS. 4 and 11) which opens out towards the tube wall 2. The sensor passage opening 28 serves to ensure good thermal contact of the temperature sensor 6 with respect to the fluid conveyed through the lumen 4 or with the tube shell wall 2. Instead of a passage opening 28, a recess can also be provided in the base body 22 in such a way that only a wall portion of the base body 22 with small wall thickness in the range between 100 μm and 1 mm remains between the temperature sensor 6 and the tube shell wall 2. Such a wall portion serves to pro-tect the temperature sensor 6.

In the embodiment with the sensor passage opening 28, the latter may be filled with a thermally conductive paste for improving thermal coupling of the temperature sensor to the tube shell wall 2.

The temperature sensor 6 may be a resistance sensor, e.g. a sensor of the Pt 100 type.

Alternatively or in addition to a temperature sensor 6 as a further sensor besides the pressure sensor 5, a sensor for measuring a fluid flow through the lumen 4 and/or an optical sensor, for example for spectroscopy of the fluid conveyed through the lumen, may also be held by the adaptation device 1a. A spectral range of a corresponding spectroscopy sensor may be in the UV wavelength range, in the VIS wavelength range, in the MIR wavelength range, and/or in the IR wavelength range.

An electrical connector 13a is accommodated in the connector receptacle 13, which in the illustrated embodiment is a two-pin female connector.

When the sensor device 1 is mounted, the tube shell wall passage opening 15 in the tube shell wall 2 of the tube 3 used in the measurement is introduced first. Then, the adaptation device 1a is positioned on the tube shell wall 2 with the positioning protrusion 25 engaging the tube shell wall passage opening 15.

The pre-assembled adaptation device 1a is equipped with the sensor unit comprising the pressure sensor 5, the temperature sensor 6, the processor 11 and the connector 13a.

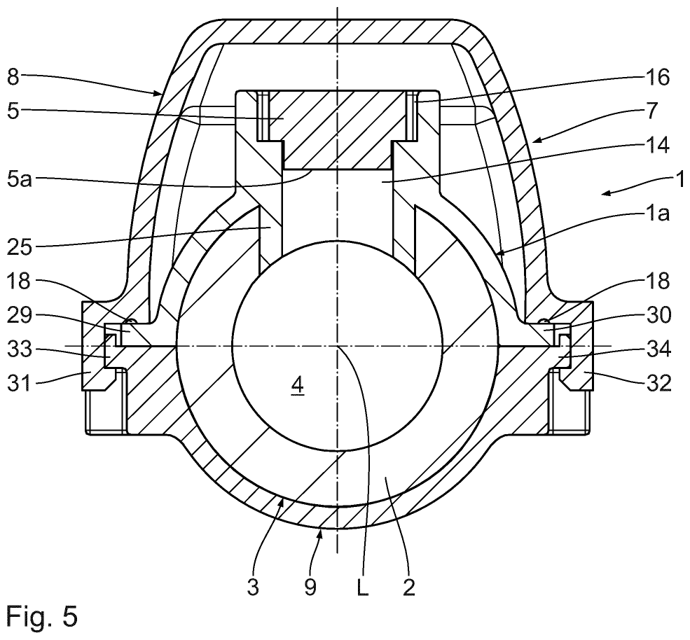

In the correctly positioned position, the lower housing part 9 and the upper housing part 8 of the sensor housing 7 are now attached to the adaptation device 1a from below and above respectively. Mounting strips 29, 30, which are integrally molded on both sides of the base body 22 of the adaptation device 1a, are clamped between clamping surfaces of the lower housing part 9 on the one hand and the upper housing part 8 on the other hand (cf. FIG. 5). The mounting strips 29, 30 form a mounting structure for supporting the adaptation device 1a on the sensor housing 7. This mounting structure also serves to seal the two housing halves 8, 9 with respect to each other. This ensures that no medium undesirably penetrates from the outside into the interior of the sensor housing 7.

The upper housing part 8 has latching hooks 31, 32 on both sides of the mounting strips 29, 30, which engage behind latching bars 33, 34 of complementary design, which are integrally molded on the lower housing part 9. The latching hooks 31, 32 are integrally molded on the upper housing part 8. This latching/clamping support of the adaptation device 1a between the housing parts 8, 9 presses the adapter housing seal 18 circumferentially against the upper housing part 8, resulting in a seal of the housing interior to the outside. In addition, this support creates a contact pressure of the adapter sealing portion 17 of the base body 22 of the adaptation device 1a against the tube shell wall 2, so that the adaptation device 1a seals against the tube shell wall 2 around the tube shell wall passage opening 15.

A seal may comply with the IP 44 standard, for example.

The plastic soft component 19 and/or the adaptation recesses 23, 24 ensure that the adaptation device 1a is adapted to the respective outer circumference of the tube shell wall 2.

After assembly of the adaptation device 1a and the housing 7, the sensor unit of the adaptation device 1a is connected to the external component via the connector 13a and the sensor device 1 is ready for measurement.

In operation of the sensor device 1, the pressure of a fluid conveyed through the lumen 4 and its temperature are measured and can be monitored thereby. The pressure measurement via the pressure sensor 5 is carried out by pressure coupling of the fluid to a pressure-sensitive sensor surface 5a via the adapter passage opening 14. A pressure measurement range of the pressure sensor 5 can be between −200 mmHg and 700 mmHg, and in particular between −50 mmHg and 300 mmHg.

The sensor device 1 is used in particular in extracorporeal membrane oxygenation (ECMO). The fluid to be measured may be respiratory air and/or blood/blood plasma. The sensors 5, 6 can be used to measure a blood pressure as well as a blood temperature.

For connection of the adaptation device 1a to the housing parts 8, 9, an adhesive can be used alternatively or additionally. In particular, an adhesive can be used which contracts during curing so that a contact pressure of the components 1a, 8 and 9 against each other is increased, thus increasing a sealing effect of the respective sealing structures.

Alternatively or additionally, a connection of the components 1a, 8 and 9 can also be made by plastic welding.

As in the embodiment shown, the adaptation device 1a can cover half the circumference of the tube shell wall 2, i.e. overlap the tube 3 with a circumferential exten-sion of 180°. Depending on the design of the adaptation device 1a and in particular also depending on the tube diameter, this circumferential overlapping area can also have a different angle in the range, for example, between 150° and 210°. The sensor housing 7 is then designed accordingly, so that in particular the housing lower part 9 engages around the remaining tube circumference.

1. An adaptation device for coupling at least one sensor to a tube shell wall of a tube for measuring a property of a fluid conveyed through the tube, comprising:
a base body,
an adapter passage opening for providing a fluid connection via a tube shell wall passage opening between a tube lumen and the sensor,
a sensor receptacle for accommodating the at least one sensor in the region of the adapter passage opening,
an adapter sealing portion extending around the adapter passage opening for sealing the adaptation device with respect to the tube shell wall in a manner surrounding the adapter passage opening, and
a mounting structure for supporting a sensor housing, the mounting structure comprising a plurality of mounting strips clamped between parts and/or between components of the sensor housing.

2. The adaptation device according to claim 1,
wherein the base body of the adaptation device is adaptable to different outer diameters of the tube shell wall.

3. The adaptation device according to claim 1,
wherein the base body in a region radially adjacent to the tube shell wall is made of a plastic soft component having a smaller Shore hardness compared to a plastic hard component of the base body, the plastic soft component disposed in a radially distal region of the base body.

4. The adaptation device according to claim 3,
wherein the plastic soft component is configured for use in an adaptable design of the base body and the adaptation device.

5. The adaptation device according to claim 2,
wherein the base body has adaptation recesses in a region radially adjacent to the tube shell wall which permit circumferential adaptation of the base body to the tube shell wall.

6. The adaptation device according to claim 1,
wherein, for adaptation to the tube shell wall, the base body has adaptation recesses in a region radially adjacent to the tube shell wall which are arranged in a circumferential direction or an axial longitudinal direction along the base body.

7. The adaptation device according to claim 1,
further comprising at least one positioning protrusion for presetting a position of the adaptation device relative to the tube shell wall by cooperating with at least one positioning recess in the tube shell wall, the at least one positioning recess having a shape complementary to the positioning protrusion.

8. The adaptation device according to claim 1,
further comprising at least one force measuring structure configured for measuring a mechanical stress acting on the base body of the adaptation device.

9. The adaptation device according to claim 1,
further comprising at least one additional sensor receptacle.

10. The adaptation device according to claim 1,
further comprising at least one connector receptacle for an electrical connector for electrical connection of the at least one sensor to an external component.

11. The adaptation device according to claim 1,
further comprising at least one sensor passage opening for coupling a another sensor to the tube shell wall of the tube.

12. A sensor device comprising:
an adaptation device for coupling a sensor to a tube shell wall of a tube for measuring a property of a fluid conveyed through the tube, the adaptation device comprised of a base body, an adapter passage, a sensor receptacle for receiving the sensor in the region of the adapter passage opening, an adapter sealing portion extending around the passage opening, and a mounting structure, the mounting structure comprising a plurality of mounting strips clamped between one of parts and components of the sensor housing,
at least one sensor accommodated in the at least one sensor receptacle of the adaptation device,
a sensor housing accommodating the at least one sensor.

13. The sensor device according to claim 12
wherein the sensor housing is designed as a multi-part housing and the mounting structure is designed such that it simultaneously effects sealing with respect to at least one of the housing parts via an adapter housing seal.

14. The adaptation device according to claim 11, wherein the another sensor comprises a temperature sensor.

15. The adaptation device according to claim 14, wherein the at least one sensor comprises a pressure sensor.

16. The adaptation device according to claim 8, wherein the at least one force measuring structure is configured for measuring a mechanical deformation of the base body.

17. The adaptation device according to claim 8, wherein the at least one sensor comprises a pressure sensor and the at least one force measuring structure is configured for at least one of calibration and correction of the pressure sensor.

18. The adaptation device according to claim 17, wherein the at least one force measuring structure comprises a strain gauge.

19. The adaptation device according to claim 3, wherein the plastic soft component is configured for sealing the base body and one of the tube shell wall and at least part of the sensor housing.

20. The adaptation device according to claim 1, further comprising at least one positioning protrusion configured to cooperate with a positioning recess in the tube shell wall to set a position of the adaptation device relative to the tube shell wall, and wherein one of the base body has a plurality of spaced apart adaptation recesses extending in a longitudinal direction of the base body configured for adapting the adaptation device different tube shell wall diameters.
